(19) United States
(12) Patent Application Publication
(10) Pub. No.: US 2025/0050081 A1
(43) Pub. Date: Feb. 13, 2025

(54) DEVICES, ASSEMBLIES, AND METHODS FOR DELIVERING A FLUID PREPARATION INTO A GASTROINTESTINAL TRACT

(71) Applicant: Rani Therapeutics, LLC, San Jose, CA (US)

(72) Inventors: Mir IMRAN, Los Altos Hills, CA (US); Paul SPEHR, San Antonio, TX (US)

(73) Assignee: Rani Therapeutics, LLC, San Jose, CA (US)

(21) Appl. No.: 18/810,246

(22) Filed: Aug. 20, 2024

Related U.S. Application Data
(63) Continuation of application No. PCT/US2023/013276, filed on Feb. 17, 2023.
(60) Provisional application No. 63/312,352, filed on Feb. 21, 2022.

(51) Int. Cl. A61M 31/00; A61M 39/08; A61M 39/22

(52) U.S. Cl. CPC A61M 31/002; A61M 39/08; A61M 39/22

(57) ABSTRACT

A delivery assembly for an ingestible device to deliver a fluid preparation into a GI lumen wall or surrounding tissue of a subject includes a housing, a piston, a needle, a conduit, and a valve member. The housing defines a piston chamber. The piston is movably disposed in the piston chamber. The needle is coupled to, or integrally formed with, the piston. The conduit is structured to direct the fluid preparation from a fluid source to the needle. The valve member has a first state and a second state. In the first state, the valve member substantially impedes a flow of the fluid preparation from the conduit to the needle. In the second state, the valve member is structured to allow the fluid preparation to flow through the conduit to the needle.

### CROSS-REFERENCE TO RELATED APPLICATIONS

This application claims the benefit of and priority to U.S. Provisional Application No. 63/312,352 filed on Feb. 21, 2022, the entire contents of which are hereby incorporated by reference herein.

### BACKGROUND

A therapeutic agent such as a drug may be administered to a subject by ingestion or through parenteral injection (e.g., subcutaneously, intramuscularly, or intravenously) to provide a desired therapeutic effect. However, these routes of administration have some disadvantages. For example, some therapeutic agents such as large (macro) molecules are not suitable for delivery by ingestion because of enzymatic breakdown of these molecules in the gastrointestinal (GI) tract of a subject. Other types of therapeutic agents may otherwise be poorly tolerated within the GI tract resulting in low systemic uptake. With parenteral injections, subjects may experience pain and inconvenience with administration which can significantly impact compliance and quality of life.

To address these disadvantages, ingestible devices have been proposed to orally deliver a solid form of a therapeutic agent into a lumen wall or surrounding tissue of the GI tract. However, solid dosage forms may be limited to relatively small dosages (e.g., a few milligrams). Thus, using solid dosage forms may be disadvantageous for therapy regimens that require higher dosages due to, for example, frequency of dosing, convenience, or cost.

Accordingly, there is a need for devices which can deliver a broad range of dosages of a therapeutic agent and can address one or more of the disadvantages associated with conventional oral and parenteral routes of administration.

### SUMMARY

Embodiments of the present disclosure relate generally to devices, assemblies, and methods for delivering a fluid preparation into a GI tract (e.g., into a GI lumen wall or surrounding tissue) of a subject. The devices and assemblies are structured to deliver up to about 400 microliters (μl) or more of a fluid preparation which may include a dosage of up to about 100 milligrams (mg) or more of at least one therapeutic agent. In this manner, the disclosed devices, assemblies, and methods can allow for efficient delivery of a broad range of dosages of therapeutic agents for most therapeutic regimens.

In one aspect, a delivery assembly for an ingestible device for delivering a fluid preparation into a GI lumen wall or surrounding tissue of a subject includes a housing, a piston, a needle, a conduit, and a valve member. The housing defines a piston chamber. The piston is movably disposed in the piston chamber. The needle is coupled to, or integrally formed with, the piston. The conduit is structured to direct the fluid preparation from a fluid source to the needle. The valve member has a first state and a second state. In the first state, the valve member substantially impedes a flow of the fluid preparation from the conduit to the needle. In the second state, the valve member is structured to allow the fluid preparation to flow through the conduit to the needle.

In another aspect, an ingestible device for delivering a fluid preparation into a GI lumen wall or surrounding tissue of a subject includes an expandable member, a container, a fluid preparation, and a delivery assembly. The container is disposed within the expandable member. The fluid preparation is disposed in the container and includes at least one therapeutic agent. The delivery assembly is coupled to, and disposed in, the expandable member. The delivery assembly includes a housing, a piston, a needle, a conduit, and a valve member. The housing defines a piston chamber. The piston is movably disposed in the piston chamber. The needle is coupled to, or integrally formed with, the piston. The conduit is structured to direct the fluid preparation from the container to the needle. The valve member is operably coupled to the conduit and has a first state and a second state. In the first state, the valve member substantially impedes a flow of the fluid preparation from the conduit to the needle. In the second state, the expandable member causes the piston to move relative to the housing to release the valve member from the conduit and allow the fluid preparation to flow from the container to the needle.

In another aspect, a method of delivering a fluid preparation into a GI lumen wall or surrounding tissue of a subject includes ingesting, by the subject, an ingestible device as described herein. The ingestible device includes an expandable member, a container, a fluid preparation, and a delivery assembly. The container is disposed within the expandable member. The fluid preparation is disposed in the container and includes at least one therapeutic agent. The delivery assembly is coupled to, and disposed in, the expandable member. The delivery assembly includes a housing, a piston, a needle, a conduit, and a valve member. The housing defines a piston chamber. The piston is movably disposed in the piston chamber. The needle is coupled to, or integrally formed with, the piston. The conduit is structured to direct the fluid preparation from the container to the needle. The valve member is operably coupled to the conduit and has a first state and a second state. In the first state, the valve member substantially impedes a flow of the fluid preparation from the conduit to the needle. In the second state, the expandable member causes the piston to move relative to the housing to release the valve member from the conduit and allow the fluid preparation to flow from the container to the needle.

In another aspect, use of an ingestible device to deliver a fluid preparation into a GI lumen wall or surrounding tissue of a subject includes ingesting, by the subject, an ingestible device as described herein. The ingestible device includes an expandable member, a container, a fluid preparation, and a delivery assembly. The container is disposed within the expandable member. The fluid preparation is disposed in the container and includes at least one therapeutic agent. The delivery assembly is coupled to, and disposed in, the expandable member. The delivery assembly includes a housing, a piston, a needle, a conduit, and a valve member. The housing defines a piston chamber. The piston is movably disposed in the piston chamber. The needle is coupled to, or integrally formed with, the piston. The conduit is structured to direct the fluid preparation from the container to the needle. The valve member is operably coupled to the conduit and has a first state and a second state. In the first state, the valve member substantially impedes a flow of the fluid preparation from the conduit to the needle. In the second state, the expandable member causes the piston to move relative to the housing to release the valve member from the conduit and allow the fluid preparation to flow from the container to the needle.

In another aspect, there are provided methods of preparing an ingestible device for delivering a therapeutic agent into a GI lumen wall or surrounding tissue of a subject, comprising filling a fluid preparation comprising a therapeutic agent into a delivery assembly as described herein.

In one or more embodiments of any of the foregoing aspects, the valve member is releasably coupled to the conduit, wherein the valve member is structured to release from the conduit in response to movement of the piston to allow the fluid preparation to flow through the conduit to the needle in the second state.

In one or more embodiments of any of the foregoing aspects, at least a portion of the conduit defines the valve member, wherein the conduit is structured to open in response to a pressure of the fluid preparation to allow the fluid preparation to flow through the conduit to the needle in the second state.

In one or more embodiments of any of the foregoing aspects, the device further includes an ingestible enclosure, wherein the expandable member and the delivery assembly are disposed in the ingestible enclosure for delivery into the GI tract of the subject.

In one or more embodiments of any of the foregoing aspects, the piston is structured to move in response to a gas pressure generated within the expandable member to advance the needle from the housing into the GI lumen wall while the valve member is in the first state.

In one or more embodiments of any of the foregoing aspects, the container is structured to expel the fluid preparation through the conduit to the needle in the second state in response to the gas pressure applied to an outer surface of the container.

In one or more embodiments of any of the foregoing aspects, the container defines a volume for containing up to about 400 μl of fluid.

In one or more embodiments of any of the foregoing aspects, the container defines a volume for containing about 50 μl to about 300 μl of fluid.

In one or more embodiments of any of the foregoing aspects, the valve member includes a cover coupled to the housing, wherein the cover defines an opening and a flexible leg extending into the opening.

In one or more embodiments of any of the foregoing aspects, the piston includes a shaft, wherein a portion of the conduit and a portion of the shaft each extend through the opening of the cover such that the shaft biases the flexible leg against the conduit to substantially impede the flow of the fluid preparation through the conduit in the first state. In one or more embodiments, the piston moves relative to the housing to cause the shaft to move away from the cover to unbias the flexible leg away from the conduit to thereby allow the fluid preparation to flow to the needle in the second state.

Alternatively, in one or more embodiments of any of the foregoing aspects, the piston includes a shaft, wherein a portion of at least one of the conduit or the shaft extends through the opening of the cover, wherein the clip is positioned outside of the piston chamber adjacent the release feature. In one or more embodiments, the piston moves relative to the housing to cause the clip to move toward the cover such that the release feature releases the clip from the conduit to thereby allow the fluid preparation to flow to the needle in the second state.

In one or more embodiments of any of the foregoing aspects, the valve member includes a clip releasably coupled to the conduit, and wherein the clip substantially impedes the flow of the fluid preparation through the conduit in the first state.

In one or more embodiments of any of the foregoing aspects, the delivery assembly further includes a cover coupled to the housing, wherein the cover defines an opening and a release feature.

In one or more embodiments of any of the foregoing aspects, the valve member includes one of a sleeve, a clip, or a band releasably coupled to the conduit such that a portion of the conduit is temporarily held in a kinked state to substantially impede the flow of the fluid preparation through the conduit in the first state.

In one or more embodiments of any of the foregoing aspects, the piston moves relative to the housing to cause tensioning of the conduit such that the sleeve, clip, or band is released from the conduit to thereby allow the fluid preparation to flow to the needle in the second state.

In one or more embodiments of any of the foregoing aspects, at least a portion of the conduit defines the valve member, wherein the at least a portion is formed from a thermoset polymer which defines a temporary seal within the conduit to substantially impede the flow of the fluid preparation through the conduit in the first state. In one or more embodiments, a pressure of the fluid preparation overcomes the temporary seal to thereby allow the fluid preparation to flow through the conduit to the needle in the second state.

In one or more embodiments of any of the foregoing aspects, the at least one therapeutic agent is one or more selected from an immunosuppressive drug, a chemotherapy drug, a central nervous system (CNS) drug, an antidiabetic drug, an enzyme replacement therapy (ERT) drug, an antibody, a hormone, insulin, an incretin or a combination thereof, or an oligonucleotide.

The foregoing general description and following detailed description are provided by way of example and are intended to provide further explanation of the disclosure as claimed, without limiting the disclosure or the claims. Other objects, advantages, and novel features will be readily apparent to those skilled in the art from the following brief description of the drawings and detailed description of the disclosure.

### BRIEF DESCRIPTION OF THE DRAWINGS

FIG. 1 illustrates an embodiment of a device for delivering a fluid preparation to a subject in block diagrammatic form.

FIG. 2 illustrates a cross-sectional view of an embodiment of the device of FIG. 1.

FIG. 3 illustrates a detail view of a portion of the device of FIG. 2.

FIG. 4 illustrates a cross-sectional view taken along line 4-4 in FIG. 3.

FIG. 5 illustrates a cross-sectional view taken along line 5-5 in FIG. 3.

FIG. 6 illustrates the device of FIG. 3 in a second state when the device has reached a desired location in the GI tract to deliver a fluid preparation.

FIG. 7 illustrates a cross-sectional view taken along line 7-7 in FIG. 6.

FIG. 8 illustrates an embodiment of a conduit and a container of the device of FIG. 2.

FIG. 9 illustrates a cross-sectional view of another embodiment of the device of FIG. 1.

FIG. 10 illustrates the device of FIG. 9 in a second state when the device has reached a desired location in the GI tract to deliver a fluid preparation.

FIG. 11 illustrates a cross-sectional view of another embodiment of the device of FIG. 1.

FIG. 12 illustrates the device of FIG. 11 in a second state when the device has reached a desired location in the GI tract to deliver a fluid preparation.

FIG. 13 illustrates a method of delivering a fluid preparation into a GI tract using the devices and assemblies of the present disclosure.

### DETAILED DESCRIPTION

Before discussing details of the devices, assemblies, and methods of the present disclosure, a few conventions are provided for the convenience of the reader.

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural references unless the context clearly dictates otherwise. Reference to an object in the singular is not intended to mean “one and only one” unless explicitly so stated, but rather “one or more.”

As used herein, a phrase in the form “A/B” or in the form “A and/or B” means (A), (B), or (A and B); a phrase in the form “at least one of A, B, or C” means (A), (B), (C), (A and B), (A and C), (B and C), or (A, B, and C).

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

The term “component” refers herein to one item of a set of one or more items that together make up a device, a composition, or a system under discussion. A component may be in a solid, powder, gel, plasma, fluid, gas, or other constitution. For example, a device may include multiple solid components which are assembled together to structure the device and may further include a fluid component that is disposed in the device. For another example, a composition may include a single component, or two or more components which are mixed together to make the composition. A composition may be in the form of a fluid, a slurry, a powder, or a solid (e.g., in a condensed or a consolidated form such as a tablet or microtablet). A device or system can include one or more compositions and/or one or more other components.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, compositions, or systems with a same design can have different actual values with respect to those characteristics. Design encompasses also variations or modifications before or after manufacture.

The term “structured” or a grammatical variation thereof (e.g., “structure” or “structuring”) refers herein to a component, device, composition, or system that is manufactured according to a concept or design or variations thereof or modifications thereto (whether such variations or modifications occur before, during, or after manufacture) whether or not such concept or design is captured in a writing.

The term “body” refers herein to an animalia body, unless the context clearly dictates otherwise.

The term “subject” refers herein to a body into which an embodiment of the present disclosure is, or is intended to be, delivered. For example, with respect to humans, a subject may be a patient under treatment of a health care professional. The terms “individual,” “subject,” and “patient” may be used interchangeably herein, and refer to any individual animalia subject (e.g., bovine, canine, feline, equine, or human). In specific embodiments, the subject, individual, or patient is a human.

The term “fluid” refers herein to a liquid or gas, and encompasses moisture and humidity. The term “fluidic environment” refers herein to an environment in which one or more fluids are present.

The term “ingest” or a grammatical variation thereof (e.g., “ingesting”, “ingestion,” or “ingested”) refers herein to taking into the stomach, whether by swallowing or by other means of depositing into the stomach (e.g., by depositing into the stomach by endoscope or depositing into the stomach via a port).

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The term “degradation rate” or a grammatical variation thereof (e.g., “rate of degradation”) refers herein to a rate at which a material degrades. A designed degradation rate of a material in a particular implementation can be defined by a rate at which the material is expected to degrade under expected conditions (e.g., in physiological conditions) at a target delivery site. A designed degradation time for a particular implementation can refer to a designed time to complete degradation or a designed time to a partial degradation sufficient to accomplish a design purpose (e.g., breach). Accordingly, for example, a designed degradation time can be specific to a component and/or specific to expected conditions at a target delivery site. A designed degradation time can be short or long and can be defined in terms of approximate times, maximum times, or minimum times.

The term “substantially” is used herein to describe and account for small variations which may result from, for example, a manufacturing or assembly process. For example, when used in conjunction with a numerical value, the terms can refer to a variation in the value of less than or equal to +10%.

The term “lumen” refers herein to the inside space of a tubular structure. Examples of lumens in a body include arteries, veins, and tubular cavities within organs.

The term “lumen wall” refers to a wall of a lumen, where the wall includes all layers from an inner perimeter to an outer perimeter of the lumen, such as, with respect to lumens in a body, the mucosa, submucosa, muscularis, serosa, and an outer wall of the lumen, with the constituent blood vessels and tissues.

The term “gastrointestinal tract” or “GI tract” refers herein to the intake/expulsion system of a body including, for example, the mouth, pharynx, esophagus, stomach, pylorus, small intestine, cecum, large intestine, colon, rectum, anus, and valves or sphincters therebetween.

The term “GI lumen” refers generally to any lumen of the GI tract (e.g., a lumen of the esophagus, stomach, small intestine, large intestine, or colon) and the term “GI lumen wall” refers to a lumen wall of a GI lumen.

As used herein, the terms “comprising”, “comprise”, “comprises”, “includes”, and “including” are intended to mean that the compositions and methods include the recited elements, but do not exclude others.

Referring generally to the Figures, disclosed herein are embodiments relating to devices, assemblies, and methods for delivering a fluid preparation into a lumen wall or surrounding tissue (e.g., a peritoneum or peritoneal cavity) of a GI tract of a subject. The devices are advantageously structured to substantially preserve one or more therapeutic agents in the fluid preparation within the GI tract for delivery into a GI lumen wall or surrounding tissue thereof. Delivery into the lumen wall or surrounding tissue can allow for systemic uptake of one or more therapeutic agents in the fluid preparation. Further, the fluid form of the preparation can allow for delivery of a broad range of dosages (e.g., up to about 100 mg or more) of one or more therapeutic agents for most therapeutic regimens. As discussed below, the disclosed devices and assemblies can deliver a variety of different types of therapeutic agents, such as macromolecules that are normally unsuitable for delivery by ingestion. In this manner, the disclosed devices, assemblies, and methods can address many of the disadvantages associated with conventional oral and parenteral routes of administration.

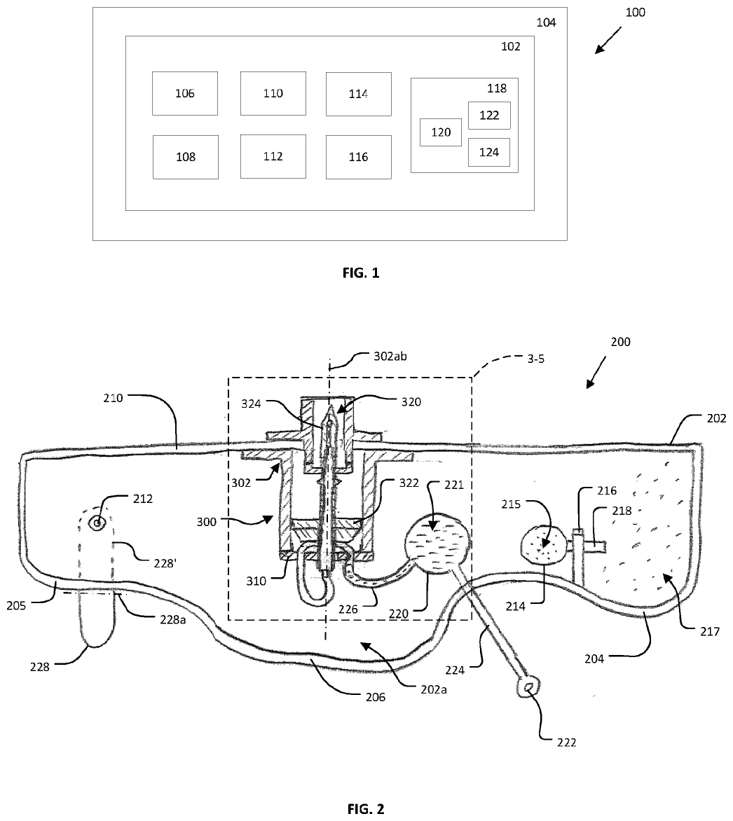

FIG. 1 illustrates in block diagrammatic form an example of a device 100 according to one or more embodiments of the present disclosure. Device 100 includes an enclosure 102, an optional outer coating 104, an expandable member 106, a release 108, a container 110, a fluid preparation 112, a conduit 114, a valve member 116, and a delivery assembly 118. Delivery assembly 118 includes at least a housing 120, a piston 122, and a needle 124.

According to a non-limiting example, enclosure 102 (or optionally enclosure 102 and/or outer coating 104) can degrade at a desired location in the GI tract (e.g., the stomach or small intestine) of a subject for delivery of fluid preparation 112, as described in more detail below. As described in more detail below, in response to at least partial degradation of enclosure 102 and/or outer coating 104, release 108 is activated to cause a chemical reaction within expandable member 106 resulting in the formation of a gas to cause expandable member 106 to expand. Expansion of expandable member 106 within the GI lumen causes delivery assembly 118 to be positioned proximate to (e.g., adjacent or in contact with) the lumen wall. When the internal pressure generated by the gas within expandable member 106 reaches a threshold value (e.g., upon full or partial expansion of expandable member 106), the pressure causes piston 122 to move relative to housing 120 to advance needle 124 from housing 120 into the GI lumen wall. The internal pressure generated by the gas is also applied against container 110 before valve member 116 is released or opened. In response to sufficient movement of needle 124 (e.g., upon needle 124 penetrating the GI lumen wall), valve member 116 is released from, or otherwise opens, conduit 114 to allow fluid preparation 112 to flow from container 110 through needle 124 and into the GI lumen wall or surrounding tissue, where one or more therapeutic agents of fluid preparation 112 can release into the subject's blood stream.

In another non-limiting example, at least a portion of conduit 114 itself may function as valve member 116, as described in more detail below. In this embodiment, conduit 114/valve member 116 may open in response to a threshold pressure applied against container 110 to allow fluid preparation 112 to flow through conduit 114 to needle 124 and into the GI lumen wall or surrounding tissue.

Enclosure 102 and optional outer coating 104, if present, are structured to allow for ingestion of device 100, and to temporarily protect the contents of device 100 from degradation within one or more portions of the GI tract of a subject. Enclosure 102 may take a variety of different forms and shapes, such as a swallowable capsule (e.g., a size 00 capsule, a size 000 capsule, or other size capsule), or any other structure that is suitable for ingestion by a subject and can house or contain one or more of the components of device 100 (e.g., at least expandable member 106, release 108, container 110, conduit 114, valve member 116, and delivery assembly 118 as discussed below). In one or more embodiments, enclosure 102 includes two or more sections coupled (e.g., press-fit) together to define enclosure 102. For example, enclosure 102 may be structured as a capsule including a first section at least partially overlapping a second section in a press-fit arrangement to define enclosure 102. The first and second sections may be detachably coupled together so as to allow for separation of the two sections. In one or more embodiments, expandable member 106, release 108, container 110, fluid preparation 112, conduit 114, valve member 116, and delivery assembly 118 are each structured to be contained within an interior of enclosure 102. Release 108 may optionally be located outside of the interior of enclosure 102, such as on an outer portion of enclosure 102.

In one or more embodiments, enclosure 102 can degrade under certain conditions. Further, different portions of enclosure 102 may be structured to degrade under different conditions or at different degradation rates depending on a target site within the GI tract for delivering fluid preparation 112. For example, a portion of, or all of, enclosure 102 may be constructed of a material that degrades in water (e.g., in the presence of water in the form of humidity or moisture in an ambient environment, such as within the body) and/or degrades when exposed to a pH level above a particular threshold or within a particular range such as a pH level associated with a target site within the GI tract for delivering fluid preparation 116.

Optional outer coating 104 optionally covers a portion of, or all of, enclosure 102. Outer coating 104 may include a single layer or multiple layers. The various layers may be formed of the same material or a combination of different materials. In one or more embodiments, outer coating 104 can degrade under certain conditions, as described above with reference to enclosure 102. An example of outer coating 104 is an enteric coating, such as an enteric coating that degrades in water at a given rate and/or degrades when exposed to solutions with a pH level above a particular threshold or within a particular range. Another example of outer coating 104 is a protective coating (e.g., wax), such as a coating which protects a portion of an outer surface of enclosure 102 from coming into contact with fluids or tissues (e.g., bodily tissue or fluids.)

In one or more embodiments, degradation of enclosure 102 and/or outer coating 104 allows fluid (e.g., bodily fluid in the stomach or in the intestine) to enter into an interior of enclosure 102/outer coating 104 to activate release 108. Alternatively, release 108 is located on an outer portion of enclosure 102 and degradation of outer coating 104 may expose release 108 on a surface of enclosure 102 so as to facilitate activation of release 108. Alternatively, release 108 may be located on a portion of enclosure 102 without outer coating 104, and release 108 may be structured to degrade at a different rate and/or under different conditions in the GI tract than enclosure 102.

Enclosure 102 and/or outer coating 104 may define one or more degradation areas for localized degradation of enclosure 102 and/or outer coating 104 so as to, for example, allow for controlled degradation and separation of enclosure 102. For example, outer coating 104 may be selectively applied only to certain areas of enclosure 102 (e.g., on the ends of enclosure 102) so as to expose a selected portion of enclosure 102 (e.g., a middle portion of enclosure 102 between the ends), thereby defining an area of enclosure 102 that can degrade at a faster rate and/or degrade sooner than other areas of enclosure 102. This controlled degradation of enclosure 102 may allow for more consistent separation of enclosure 102 to thereby allow for delivery of fluid preparation 112 into the GI lumen wall from delivery assembly 118.

Expandable member 106 is a flexible and adjustable structure. In one or more embodiments, expandable member 106 is structured to expand from a collapsed state (e.g., folded, rolled, flattened) to an expanded state within a desired location of a GI lumen for delivery of fluid preparation 112. Expandable member 106 may have a variety of different shapes, sizes, and configurations for being temporarily stored in enclosure 102 and for being deployed within a lumen of the GI tract of a subject. Expandable member 106 may be structured as a balloon, a bellows/accordion structure, or any other structure that can adjust from a collapsed state to an expanded state.

Expandable member 106 defines an interior volume for containing various components of device 100. For example, expandable member 106 may include (e.g., contain) one or more reactants that are temporarily separated from each other within the interior volume. The interior volume is structured to facilitate a chemical reaction caused by mixing the reactants together (e.g., in response to activation of release 108) to form a gas to cause expansion of expandable member 106. Expandable member 106 may further include a structure for deflating expandable member 106 upon delivery of fluid preparation 112 to facilitate excretion of expandable member 106 from a subject. Expandable member 106 is structured to stretch a defined amount without perforation upon expansion.

Expandable member 106 may be formed from one or more materials. Examples of suitable materials for expandable member 106 include hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof. Expandable member 106 may include one or more layers of material. Expandable member 106 may be a monolithic structure. In other embodiments, expandable member 106 may be composed of one or more sections that are coupled (e.g., sealed or sewn) together.

Release 108 is a chemical, mechanical, electrical, electro-mechanical, electro-chemical, chemo-mechanical, or electro-mechanical-chemical structure. Release 108 is structured to cause expansion of expandable member 106 upon activation of release 108. In one or more embodiments, release 108 is structured to be activated (e.g., degrade, release, move, open) in response to a condition in the GI tract. For example, release 108 may be structured to degrade in water, such that release 108 may degrade upon contact with fluid in the GI tract. For another example, release 108 may be structured to degrade at or above a particular pH level or within a range of pH levels associated with a location in the GI tract (e.g., a pH in the stomach, a pH in the intestine.) In these and other embodiments, release 108 may be made from a biodegradable material, such as an enteric material. Additionally or alternatively, release 108 may be structured as a latch, a clip, a cover, a plug, a coating, or any other structure that moves, opens, or otherwise releases in response to a condition in the GI tract.) Release 108 may be formed from a single material or a combination of materials. Release 108 may include one or more components. In embodiments in which a plurality of components are included in release 108, the components may be co-located (e.g., co-axially) or may be physically separated from each other.

Container 110 is a flexible structure that defines an interior volume for containing fluid preparation 112. The interior volume may contain up to about 400 μl or more of fluid, including 400 μl of fluid. For example, the interior volume may contain about 50 μl to about 300 μl of fluid, such as 50 μl, 100 μl, 150 μl, 200 μl, 250 μl, or 300 μl of fluid, or any value therebetween, or may contain 300 μl, 350 μl, or 400 μl of fluid, or any value therebetween. Container 110 is further structured to be contained within an interior of expandable member 106. Container 110 may have any suitable shape, such as spherical, cuboidal, ellipsoidal, or other shape. Container 110 is further structured to expel fluid preparation 112 from the interior volume in response to a pressure (e.g., from a gas generated within expandable member 106) applied to an outer surface of container 110. Container 110 may be formed from, or otherwise include, a flexible or pliable material to allow for deformation (e.g., constriction by squeezing) of container 110 to expel fluid preparation 112. Examples of suitable materials for container 110 include a flexible polymeric material (e.g., polyethylene terephthalate (PET)), or other materials or combinations of materials. Container 110 may be formed from a single material or a combination of materials. Further, container 110 may include one or more layers of a material. Container 110 may be a monolithic structure. In other embodiments, container 110 may be composed of multiple sections coupled (e.g., sealed) together to define container 110. Container 110 may include conduit 114 integrally formed therewith for directing fluid preparation 112 to needle 124. In other embodiments, conduit 114 is coupled (e.g., press-fit, adhered) to container 114. Container 110 may include one or more components. In embodiments in which a plurality of components are included in container 110, the components may be co-located or may be physically separated from each other.

Fluid preparation 112 is a preparation including one or more components where the preparation is intended for a therapeutic, diagnostic, or other biological purpose. Fluid preparation 112 is in a fluid form, such as a liquid, a slurry, a gel, a suspension (including a colloidal suspension), a gas, a powder, or any combination thereof. Fluid preparation 116 includes one or more components including one or more therapeutic agents, such as a protein, a peptide, a polypeptide, an antibody, a drug (e.g., one or more selected from immunosuppressive drugs (e.g., adalimumab), chemotherapy drugs, central nervous system (CNS) drugs (e.g., antiparkinson agents, antiemetic agents), antidiabetic drugs (e.g., metformin), enzyme replacement therapy (ERT) drugs), a hormone (e.g., parathyroid hormone (PTH), follicle stimulating hormone (FSH), and analogues thereof), insulin, an incretin or a combination thereof (e.g., GLP-1, GLP-2, GIP, glucagon, PYY, and analogues thereof)), an oligonucleotide (e.g., antisense oligonucleotides (ASO), RNA interference (RNAi), aptamer RNAs), a DNA or SiRNA transcript, a cell, a cytotoxic agent, a vaccine or other prophylactic agent, a nutraceutical agent, a vasodilator, or a vasoconstrictor, a delivery enhancing agent, a delay agent, an excipient, a diagnostic agent, or a substance for cosmetic enhancement. Fluid preparation 112 may include a therapeutically effective amount of one or more therapeutic agents as well as suitable amounts of other components (e.g., excipients) to achieve a desired therapeutic effect in a subject.

Conduit 114 is a mechanical structure for directing fluid preparation 112 from container 110 to needle 124. Conduit 114 may be coupled to, or integrally formed with, container 110. Conduit 114 may form part of delivery assembly 118. For example, conduit 114 may be coupled to, or integrally formed with, a shaft of piston 122. Conduit 114 is structured to direct fluid preparation 112 from container 110 to delivery assembly 118 for delivery of fluid preparation 122 into a GI lumen wall or surrounding tissue thereof. At least a portion of, or all of, conduit 114 is flexible or deformable. Conduit 114 may be formed from one or more materials. Suitable materials for conduit 114 include, for example, silicone (e.g., silicone tubing), a thermoset polymer (e.g., polyimide tubing), or other material or combinations of materials. In one or more embodiments, conduit 114 is further structured to interface with valve member 116 to selectively control a flow of fluid preparation 112 through conduit 114. In other embodiments, at least a portion of conduit 114 itself may define valve member 116 (e.g., where conduit 114 includes a section formed from a thermoset polymer, such as polyimide.) Conduit 114 may include one or more components. In embodiments in which a plurality of components are included in conduit 114, the components may be co-located or may be physically separated from each other.

Valve member 116 is a chemical, mechanical, electrical, electro-mechanical, electro-chemical, chemo-mechanical, or electro-mechanical-chemical structure. Valve member 116 is structured to interface with conduit 114 to selectively control a flow of fluid preparation 112 from container 110 to delivery assembly 118. In one or more embodiments, valve member 116 may form part of delivery assembly 118. For example, valve member 116 may include a cover that is coupled to housing 120. The cover may include a biasing feature for interfacing with conduit 114 to function as a pinch-valve. Additionally or alternatively, the cover may include a release feature for engaging with a valve clip that is releasably coupled to conduit 114. The release feature and the valve clip may cooperatively define valve member 116. Additionally or alternatively, conduit 114 may be held in a kinked state by valve member 116, which may be structured as a sleeve, a band, a clip, or other structure releasably coupled to conduit 114. In these embodiments, movement of piston 122 relative to housing 120 can cause valve member 116 to release from, or otherwise open, conduit 114 to allow fluid preparation 112 to flow from container 110 to needle 124 for delivery into the GI lumen wall or surrounding tissue thereof.

Additionally or alternatively, conduit 114 may include at least a portion formed from a thermoset polymer (e.g., polyimide) such that conduit 114 itself can function as valve member 116. The thermoset polymer portion may be heated and compressed to define a temporary seal within conduit 114 so as to substantially prevent a flow of fluid preparation 112 therethrough. In this embodiment, a pressure of fluid preparation 112 can overcome the temporary seal within conduit 114 to open (partially or fully) conduit 114 and expel fluid preparation to allow fluid preparation 112 to flow from container 110 to needle 124 for delivery into the GI lumen wall or surrounding tissue thereof. Valve member 116 may include one or more components. In embodiments in which a plurality of components are included in valve member 116, the components may be co-located or may be physically separated from each other.

Delivery assembly 118 is a chemical, mechanical, electrical, electro-mechanical, electro-chemical, chemo-mechanical, or electro-mechanical-chemical structure. In one or more embodiments, delivery assembly 118 includes housing 120, piston 122, and needle 124. As explained above, valve member 116 and conduit 114 may also form part of delivery assembly 118.

Housing 120 defines a piston chamber for movably receiving piston 122. Housing 120 further includes a structure for temporarily retaining needle 124 therein. Housing 120 is structured to be coupled to, and is in fluid communication with, expandable member 106. In one or more embodiments, housing 120 may include a cover that can function as part of (or may define) valve member 116.

Piston 122 is movably (e.g., slidably) disposed in housing 120. Piston 122 may include a shaft coupled to, or integrally formed, with piston 122. Conduit 114 may be coupled to, or integrally formed with, an end of the shaft. Needle 124 may be coupled to, or integrally formed with, an opposite end of the shaft. The shaft may be at least partially hollow to permit a flow of fluid preparation 112 from conduit 110 to needle 124.

Needle 124 is structured to penetrate into the GI lumen wall or surrounding tissue thereof and to deliver fluid preparation 112 into the lumen wall or surrounding tissue. Needle 124 includes an opening for discharging fluid preparation 112. Needle 124 may be at least partially, or may be fully, degradable such that needle 124 can degrade within the GI lumen after delivery of fluid preparation 112. Suitable materials for needle 124 may include, for example, polyethyleneoxide (PEO), magnesium, a polymeric material, or other material or combinations of materials. In other embodiments, needle 124 may be substantially non-degradable or may include substantially non-degradable portions. In any embodiments, and particularly in embodiments where the needle is substantially non-degradable, delivery assembly 118 may be equipped with a return spring or other structure to permit retraction of needle 124 from the GI lumen wall within housing 120 to prevent injury to a subject.

In one or more embodiments, delivery assembly 118 is structured such that when the internal pressure generated by the gas within expandable member 106 reaches a threshold value, the gas pressure causes piston 122 to move within housing 120 to advance needle 124 out of housing 120 and into a GI lumen wall or surrounding tissue thereof. When valve member 116 is released from, or otherwise opens, conduit 114 (e.g., upon needle 124 penetrating the GI lumen wall), delivery assembly 118 is further structured to deliver fluid preparation 112 from needle 124 into the GI lumen wall or surrounding tissue. Device 100 may include one or more injector assemblies 118. Delivery assembly 118 may include one or more components. In embodiments in which a plurality of components are included in delivery assembly 118, the components may be co-located or may be physically separated from each other.

One or more components of device 100 (e.g., capsule 102, outer coating 104, expandable member 106, release 108, container 110, conduit 114, valve member 116, housing 120, piston 122, needle 124) may be formed from, or otherwise include, one or more biodegradable materials to facilitate degradation of such components to, for example, allow for passage through the remainder of the intestinal tract of a subject after delivery of fluid preparation 112. Examples of biodegradable materials that may be suitable for use with various components of ingestible device 100 include, for example, hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof.

The preceding description is an overview of ingestible device 100. The following description with reference to FIGS. 2-10 relates to examples of various features and aspects of device 100. These examples are illustrative of, and not limiting on, device 100.

Referring to FIG. 2, a cross-sectional view of a device 200 (an embodiment of device 100) is illustrated in an unfolded and/or unrolled state. Device 200 is illustrated without enclosure 102 and/or outer coating 104. However, it should be appreciated that device 200 may be folded, rolled, and/or otherwise manipulated to be disposed within enclosure 102 and/or outer coating 104 to allow for ingestion of device 200 and subsequent delivery into the GI tract.

Device 200 includes an expandable member in the form of a balloon 202 (an embodiment of expandable member 106), a deflation valve 212, a reactant reservoir 214 containing a first reactant 215, a release 216 (an embodiment of release 108), a second reactant 217, a container 220 (an embodiment of container 110), a fluid preparation 221 (an embodiment of fluid preparation 112) disposed in container 220, a conduit 226 (an embodiment of conduit 114), and a delivery assembly 300 (an embodiment of delivery assembly 118.)

Still referring to FIG. 2, balloon 202 includes an inflator section 204, a deflator section 205, a lower section 206, and an elongated section 210 extending between inflator section 204 and deflator section 205. Lower section 206 extends downward (in the orientation shown in FIG. 2) between inflator section 204 and deflator section 205. The various sections of balloon 202 cooperatively define an interior 202a for containing various components of device 200. As discussed below, balloon 202 is structured to inflate at a desired location within a lumen of the GI tract (e.g., stomach, intestine) in response to the formation of a gas from a chemical reaction between first reactant 215 and second reactant 217 within interior 202a. In this way, balloon 202 can facilitate delivery of fluid preparation 221 from delivery assembly 300 into the GI lumen wall or surrounding tissue thereof.

Balloon 202 has a size and shape to occupy a space in a GI lumen upon inflation of balloon 202 to allow for delivery of fluid preparation 221 into the GI lumen wall. For example, upon inflation of balloon 202, an outer periphery of balloon 202 (e.g., an outer periphery of lower section 206 and elongated section 210) pushes against a surface of the lumen wall. The pressure exerted by balloon 202 is sufficient to temporarily hold balloon 202 relative to the lumen wall for delivery of fluid preparation 221. Depending on an inner circumference of the lumen delivery site, lower section 206 may remain partially folded, or may extend fully, when balloon 202 is inflated. For example, if the lumen is relatively large and there is no obstruction to resist expansion of balloon 202, then balloon 202 would assume a fully inflated configuration with lower section 206 fully extended (as shown in FIG. 2.) If, however, the lumen is relatively small such that the inner lumen circumference is less than a maximum dimension of the fully inflated balloon 202, then lower section 206 would remain partially folded. In this way, balloon 202 can self-adjust to the size of a GI lumen to hold balloon 202 in position for delivery of fluid preparation 221, such that the same balloon 202 can be used for a broad range of lumen sizes (e.g., different inner circumferences.)

Deflation valve 212 is structured to cause deflation of balloon 202 upon completion of delivery of fluid preparation 221 into the GI lumen wall or surrounding tissue thereof. In this way, deflation valve 212 can facilitate passage of balloon 202 through the remainder of the GI tract to exit the anus of the subject. In the embodiment shown, deflation valve 212 is structured as a degradable plug which temporarily covers an opening leading into interior 202a. The degradable plug may be structured to degrade in response to contact with fluid in the GI tract (e.g., bodily fluid) to thereby allow gas contained in interior 202a to exit through the opening. For example, deflation valve 212 may be formed from, or include, an enteric material. Balloon 202 may include an optional flap 228 which can temporarily cover deflation valve 212 until balloon 202 is inflated to thereby prevent premature activation (e.g., degradation) of deflation valve 212. For example, flap 228 may be temporarily held (e.g., adhered, tacked, or otherwise held) in a folded position 228′ about a flap folding axis 228a. The expansion of balloon 202 can cause flap 228 to unfold from the folded position 228′ to expose deflation valve 212 and allow fluid in the GI tract to reach deflation valve 212 and cause its degradation, thereby providing an opening for gas to exit through to permit deflation.

Deflation valve 212 is shown located on deflator section 205, but deflation valve 212 may be located elsewhere on expandable member 202 according to other embodiments. Further, balloon 202 may include more than one deflation valve 212. Deflation valve 212 may be structured differently than the embodiment shown in FIG. 2. For example, in other embodiments, deflation valve 212 may be structured as a degradable and/or movable cover disposed over an opening on balloon 202. In these embodiments, degradation and/or movement of the cover away from the opening can cause gas to exit from interior 202a through the opening, thereby permitting deflation.

Reactant reservoir 214 is disposed within interior 202a and is structured to hold first reactant 215 therein and to temporarily prevent first reactant 215 from contacting second reactant 217, which is separately disposed within interior 202a. First reactant 215 may be, for example, citric acid. Second reactant 217 may be, for example, a carbonate, such as potassium bicarbonate. In other embodiments, first reactant 215 and second reactant 217 may be other types of reactants (e.g., an acid and a base) which when mixed result in the formation of a gas sufficient to inflate balloon 202. Second reactant 217 is shown disposed within interior 202a at inflator section 204 near first reactant 215. In other embodiments, first reactant 215 and second reactant 217 may be contained in other areas of balloon 202 so long as they are temporarily separated from each other.

Reactant reservoir 214 defines an interior volume for containing first reactant 215. Reactant reservoir 214 may take a variety of different forms and shapes, such as a balloon or other structure. Reactant reservoir 214 is in selective fluid communication with interior 202a via reactant conduit 218 and release 216. Release 216 is coupled to reactant conduit 218 such that upon activation (e.g., degradation) of release 216, first reactant 215 can exit from reactant reservoir 214 into interior 202a via reactant conduit 218. For example, release 216 may be in the form of a degradable plug which blocks an interior portion of reactant conduit 218 to temporarily prevent first reactant 215 from entering interior 202a. When fluid in the GI tract contacts release 216 (e.g., upon degradation of enclosure 102 and/or outer coating 104), release 216 can subsequently degrade to allow reactant conduit 218 to discharge first reactant 215 into interior 202a.

In other embodiments, device 200 includes a clip, a band, or other structure for holding a portion of balloon 202 in such a manner so as to temporarily define separate chambers within interior 202a for separately containing first reactant 215 and second reactant 217, respectively. For example, a portion of balloon 202 may be pinched or compressed by a degradable clip or band to temporarily define the separate chambers within interior 202a. The chambers may be substantially sealed from each other to substantially prevent first reactant 215 and second reactant 217 from mixing. The clip or band may be located on an outer portion of balloon 202 such that exposure to fluid in the GI tract (e.g., upon degradation of enclosure 102 and/or outer coating 104) can cause degradation of the clip or band and subsequent release from balloon 202. Upon release of the clip or band or like structure from balloon 202, the separate chambers are no longer substantially sealed from each other thereby allowing first reactant 215 to mix with second reactant 217 within interior 202a.

Combining first reactant 215 with second reactant 217 within interior 202a causes a chemical reaction resulting in the formation of a gas (e.g., CO₂.) The gas causes balloon 202 to expand to an inflated state within a GI lumen resulting in substantial alignment of elongated section 210 with a surface of the GI lumen wall. Substantial alignment of elongated section 210 relative to the GI lumen wall can, advantageously, help to facilitate delivery of fluid preparation 221 from delivery assembly 300 into the GI lumen wall or surrounding tissue thereof.

Still referring to FIG. 2, container 220 defines a reservoir for holding fluid preparation 221 therein. Container 220 is structured to expel fluid preparation 221 to delivery assembly 300 via conduit 226 in response to a pressure applied to an outer surface of container 220. Container 220 is disposed within interior 202a and is coupled (e.g., heat sealed) to balloon 202. For example, during the assembly process of device 200, a fill port 222 extends from container 220 by a fill conduit 224. The fill port 222 and fill conduit 224 are structured to allow for initial filling of container 220 with a volume (e.g., up to about 400 μl or more, including a volume in a range of about 50 μl to about 300 μl, such as 50 μl, 100 μl, 150 μl, 200 μl, 250 μl, or 300 μl of fluid, or any value therebetween, or a volume of 300 μl, 350 μl, or 400 μl, or any value therebetween) of fluid preparation 221 using, for example, a syringe or other device. Upon completion of filling container 220, balloon 202 can be sealed (e.g., via heat sealing) along a periphery of balloon 202 including a portion of fill conduit 224. In this way, a section of fill conduit 224 within interior 202a is substantially sealed to prevent fluid preparation 221 from exiting container 220 through fill conduit 224. Further, the sealing of balloon 202 along fill conduit 224 fixes the position of container 220 relative to balloon 202. The section of fill conduit 224 including fill port 222 located outside of the periphery of balloon 202 can be removed from balloon 202 after sealing.

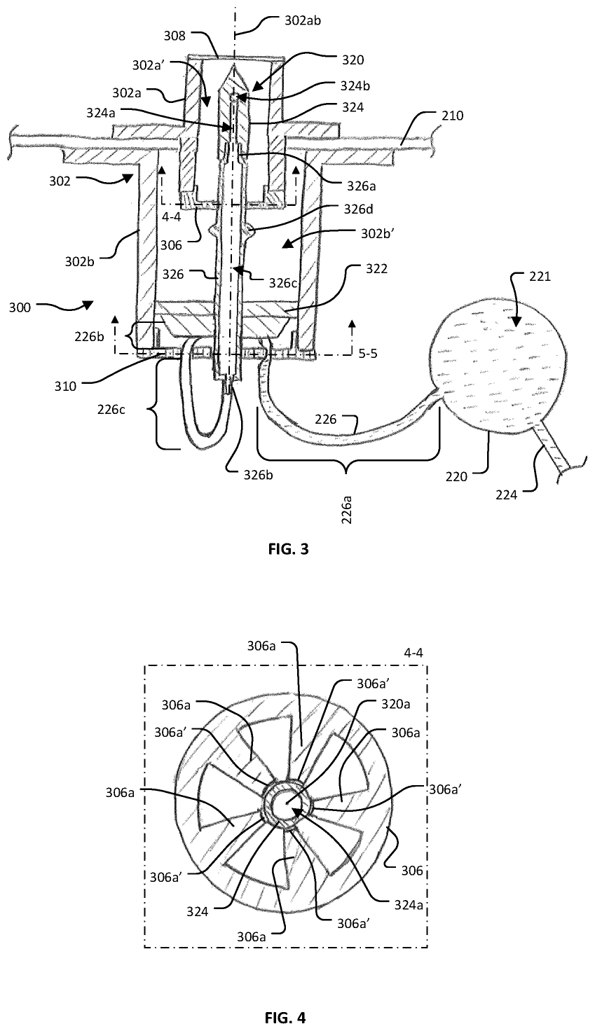

In the embodiment shown, container 220 has a spherical shape. In other embodiments, container 220 may have other shapes, such as cuboidal, ellipsoidal, or any other shape. Container 220 may be structured as a balloon or other bladderlike structure. Container 220 is formed, at least in part, from a flexible or pliable material to permit deformation (e.g., constriction by squeezing) of container 220 to expel fluid preparation 221 from container 220 to delivery assembly 300. For example, the gas formed within interior 202a results in an internal pressure that is applied against an outer surface of container 220. The pressure is sufficient to cause squeezing of container 220 to thereby expel fluid preparation 221 from container 220 when valve member 310 is released from conduit 226, the details of which are discussed below with respect to FIGS. 3-12.

Conduit 226 is coupled to, or integrally formed with, container 220. Conduit 226 is structured to direct fluid preparation 221 to delivery assembly 300 for delivery of fluid preparation 221 into the GI lumen wall or surrounding tissue thereof. Conduit 226 is shown as a substantially flexible tube (e.g., silicone tube, polyimide tube) in the embodiment of FIG. 2, but it should be appreciated that conduit 226 may include substantially non-flexible sections according to other embodiments. In the embodiment shown, conduit 226 is structured to interface with a valve member in the form of a cover 310 (an embodiment of valve member 116) to selectively control a flow of fluid preparation 221 from container 220 to delivery assembly 300, as discussed below with respect to FIGS. 3-6.

Still referring to FIG. 2, delivery assembly 300 is coupled to balloon 202 and is in separate fluid communication with interior 202a and container 220. For example, the gas that pressurizes balloon 202 can pass through one or more openings of cover 310 to apply pressure against a piston 322 of delivery assembly 300. When the pressure reaches a threshold (e.g., upon full or partial inflation of balloon 202), the gas moves piston 322 and advances a needle 324 of delivery assembly 300 into the GI lumen wall or surrounding tissue thereof. The gas that pressurizes balloon 202 is also applied against an outer surface of container 220 for expelling fluid preparation 221 through conduit 226 and into and through needle 324. However, as discussed below, cover 310 is structured to interface with conduit 226 to substantially impede a flow of fluid preparation 221 through conduit 226 to needle 324 until needle 324 has traveled a sufficient distance. Upon sufficient travel of needle 324 (e.g., upon needle 324 penetrating the GI lumen wall), cover 310 is further structured to release conduit 226 to allow fluid preparation 221 to flow through conduit 226 to needle 324 for delivery into the GI lumen wall or surrounding tissue thereof.

Referring to FIG. 3, a detail view of a portion of device 200 including a portion of balloon 202 (e.g., elongated section 210) and delivery assembly 300, which is illustrated in an unactuated state. Delivery assembly 300 includes a housing 302, a release mechanism 306, an upper seal 308, cover 310, piston 322, needle 324, and a shaft 326. Piston 322, needle 324, and shaft 326 may cooperatively define a piston-needle assembly 320. As discussed below, piston-needle assembly 320 is structured to move axially relative to housing 302 along a longitudinal axis 302ab defined by housing 302 to penetrate the GI lumen wall and deliver fluid preparation 221.

Housing 302 is collectively defined by an upper housing section 302a and a lower housing section 302b. Upper housing section 302a may be coupled (e.g., heat staked, adhered) to lower housing section 302b with a portion of balloon 202 (e.g., a portion of elongated section 210) disposed therebetween. In other embodiments, upper housing section 302a and lower housing section 302b may define a unitary (one-piece) structure. Upper housing section 302a extends outwardly from elongated section 210 and is structured to temporarily house needle 324 therein. Upper housing section 302a has a substantially cylindrical shape and defines a needle chamber 302a′ for containing needle 324 therein, although other shapes are contemplated according to other embodiments. Upper housing section 302a and lower housing section 302b cooperatively define longitudinal axis 302ab. Seal 308 is coupled (e.g., adhered) to an upper portion of upper housing section 302a to substantially prevent fluid in the GI tract from entering into needle chamber 302a′ until needle 324 pierces seal 308. Seal 308 may be formed from a penetrable material (e.g., aluminum foil) to allow needle 324 to pierce through seal 308. In this way, seal 308 and upper housing section 302a can cooperatively define a substantially sterile environment within needle chamber 302a′ for containing needle 324 before delivery of fluid preparation 221 into the GI lumen wall.

Needle 324 is temporarily disposed within needle chamber 302a′. Needle 324 includes a tapered end (e.g., pointed end) that is structured to pierce though seal 308 and penetrate the GI lumen wall or surrounding tissue thereof in response to sufficient axial movement of piston 322. Needle 324 defines a needle channel 324a extending from an opposite end of needle 324 to a needle opening 324b located adjacent the tapered end for discharging fluid preparation 221. In other embodiments, needle 324 may include an opening at the pointed end for discharging fluid preparation 221. Needle 324 is coupled (e.g., press-fit) to, or integrally formed with (e.g., insert molded), shaft 326. Needle 324 is at least partially, or may be fully, degradable such that needle 324 can substantially degrade within the GI lumen wall (or other location in the GI tract) upon delivery of fluid preparation 221. For example, needle 324 may be formed from, or otherwise include, polyethyleneoxide (PEO), magnesium, or other degradable material or combinations of materials.

Release mechanism 306 (e.g., actuator, trigger) is coupled (e.g., press-fit, snap-fit, adhered) to, or integrally formed with, a lower portion of upper housing section 302a. Release mechanism 306 is structured to interface with shaft 326 to prevent advancement of needle 324 until sufficient pressure is generated by the gas within interior 202a. For example, FIG. 4 illustrates a cross-sectional view of release mechanism 306 taken along line 4-4 in FIG. 3. As shown in FIG. 4, release mechanism 306 has a substantially circular cross-sectional shape and includes one or more fingers 306a extending radially inward toward longitudinal axis 302ab. Each of the fingers 306a extends radially inward in a cantilevered manner from a circumferential edge of release mechanism 306 to a free end 306a′ located adjacent shaft 326. Fingers 306a are structured to interface with a protrusion 326d of shaft 326 (shown in FIG. 3) via an interference (e.g., overlapping) condition to substantially prevent needle 324 from piercing seal 308 until sufficient pressure is generated within interior 202a. When the internal pressure reaches a threshold (e.g., upon full or partial inflation of balloon 202), protrusion 326d overcomes the interference condition with fingers 306a by causing free ends 306a′ to deflect away from shaft 326 to thereby allow shaft 326 to move axially along longitudinal axis 302ab toward upper housing section 302a, such that needle 324 pierces seal 308 and advances into the GI lumen wall.

Referring to FIG. 3, lower housing section 302b extends within interior 202a and defines a piston chamber 302b′ for movably receiving piston 322. Piston 322 is disposed within piston chamber 302b′ and is movably (e.g., slidably) coupled to an inner surface of lower housing section 302b which defines piston chamber 302b′. Piston 322 may include one or more seals for interfacing with the inner surface of lower housing section 302b to create a substantially fluid-tight seal within at least a portion of piston chamber 302b′.

Shaft 326 is coupled to (e.g., press-fit, adhered), or integrally formed (e.g., insert molded) with, piston 322. In the embodiment shown, shaft 326 is structured as a substantially cylindrical member with a generally elongated configuration, although other shapes and configurations are contemplated according to other embodiments. Shaft 326 extends from a first end 326a to a second end 326b. First end 326a is coupled to (e.g., press-fit, adhered, insert molded), or integrally formed with, needle 324. Second end 326b is coupled to (e.g., via a hose barb, clamp), or integrally formed with, conduit 226. Piston 322 is located between first end 326a and second end 326b. Shaft 326 further defines an inner shaft channel 326c for directing fluid preparation 221 from conduit 226 to needle channel 324a. As shown in FIG. 3, shaft channel 326c extends the entire length of shaft 326. In other embodiments, shaft channel 326c extends a portion of the length of shaft 326. In the position shown in FIG. 3, protrusion 326d is located on shaft 326 below (e.g., adjacent) release mechanism 306 outside of upper housing section 302a. As discussed above, protrusion 326d interfaces with fingers 306a of release mechanism 306 to substantially prevent advancement of needle 324 through seal 308 until the internal pressure provided by the gas within interior 202a reaches a threshold value. For example, the threshold pressure may be associated with a fully inflated state of balloon 202 to ensure substantial alignment of elongated section 210 with the GI lumen wall before advancing needle 324 into the lumen wall.

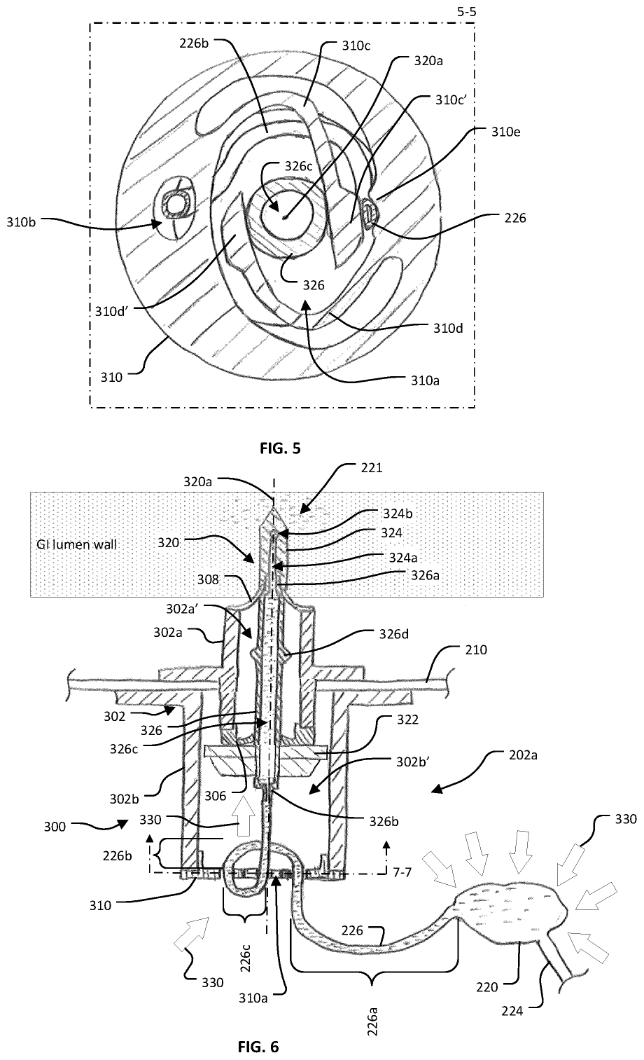

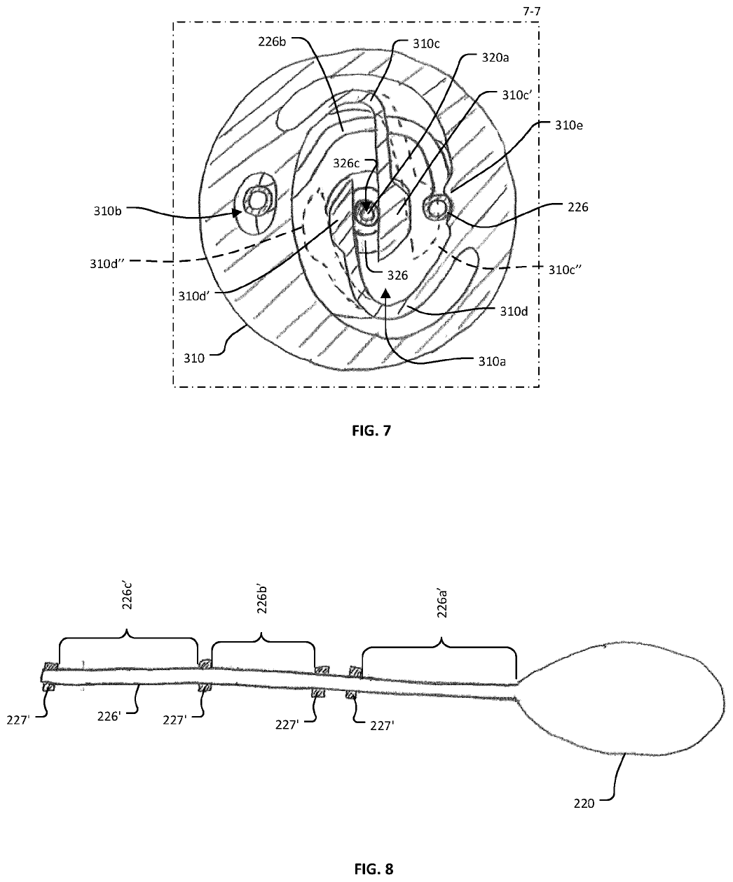

Cover 310 is coupled (e.g., press-fit, snap-fit, adhered) to, or integrally formed with, lower housing section 302b. In the embodiment shown, cover 310 includes a biasing feature which interfaces with conduit 226 to function as a pinch-valve for selectively controlling a flow of fluid preparation 221 to needle 324. Cover 310 is further structured to allow the gas that pressurizes balloon 202 to apply a pressure against a surface of piston 322, to thereby move piston 322 within piston chamber 302b′ and advance needle 324 through seal 308. For example, FIG. 5 illustrates a cross-section of cover 310 taken along line 5-5 in FIG. 3. As shown in FIG. 5, cover 310 has a generally circular cross-sectional shape, although other shapes and configurations are contemplated according to other embodiments. Cover 310 includes a first opening 310a and a second opening 310b. Cover 310 further includes a first leg 310c and a second leg 310d each extending into first opening 310a. First leg 310c and second leg 310d are flexible members that are structured to deflect between a biased position (e.g., a first state), shown in FIGS. 3-5, and an unbiased position (e.g., a second state), shown in FIGS. 6-7, within first opening 310a to selectively control a flow of fluid preparation 221 through conduit 226 to needle 324. It is worth noting that first leg 310c functions as the biasing feature for controlling fluid flow through conduit 226, whereas second leg 310d is an optional feature which may help to ensure axial alignment of shaft 326 relative to longitudinal axis 302ab, as discussed below.

As shown in FIG. 5, first leg 310c has a generally arcuate shape and extends from an upper left-side portion of cover 310 to a middle right side portion of cover 310 within first opening 310a. First leg 310c terminates at a free end defined by a first foot 310c′ within first opening 310a. Second leg 310d is the mirror image of first leg 310c. Second leg 310d has a generally arcuate shape and extends from a lower right-side portion of cover 310 to a middle left-side portion of cover 310 within first opening 310a. Second leg 310d terminates at a free end defined by a second foot 310d′ within first opening 310a adjacent first foot 310c′. First leg 310c and second leg 310d are each biased laterally away in opposite directions from longitudinal axis 302ab by shaft 326 in the first state shown in FIG. 5. Cover 310 includes an optional depression 310e located adjacent first foot 310c′ for engaging with a portion of conduit 226. Depression 310e may help to maintain a position of conduit 226 relative to first foot 310c′.

Still referring to FIG. 5, a portion of shaft 326 extends through first opening 310a between first foot 310c′ and second foot 310d′ along longitudinal axis 302ab such that first leg 310c and second leg 310d are biased laterally away from longitudinal axis 302ab in opposite directions by shaft 326. First foot 310c′ and second foot 310d′ apply a biasing force on opposite sides of shaft 326, which may help to maintain an axial position (along longitudinal axis 302ab) of shaft 326 relative to cover 310. Conduit 226 extends through first opening 310a between depression 310e and an opposite side of first foot 310c′. First foot 310c′ is biased by shaft 326 against conduit 226 such that conduit 226 is locally constricted (e.g., pinched) by first foot 310c′ to substantially impede a flow of fluid preparation 221 through conduit 226 to needle 324 in the first state shown in FIGS. 3-5. In other words, first foot 310c′ effectively functions as a pinch-valve for controlling a flow of fluid preparation 221 through conduit 226.

As discussed below with respect to FIG. 6 and FIG. 7, which illustrates first leg 310c in the second state, sufficient movement of piston-needle assembly 320 causes first foot 310c′ to move laterally away from conduit 226 to an unbiased (relaxed) position such that conduit 226 is no longer constricted by first foot 310c′. Further, the spacing between first foot 310c′ and second foot 310d′ in the unbiased position allows for substantially unobstructed movement of third conduit section 226c through first opening 310a to thereby permit axial movement of piston-needle assembly 320. In this manner, fluid preparation 221 can flow through conduit 226 and shaft 326 to needle 324 for delivery into the lumen wall in the second state.

As shown in FIGS. 3 and 5, conduit 226 includes a first conduit section 226a extending from container 220 to cover 310, a second conduit section 226b extending within second housing section 302b, and a third conduit section 226c extending from cover 310 to shaft 326. Second conduit section 226b is routed from first conduit section 226a through first opening 310a at least partially around shaft 326 within second housing section 302b, and through second opening 310b to third conduit section 226c. Third conduit section 226c is structured to move with piston-needle assembly 320 along longitudinal axis 302ab.

To assist with assembly and routing of conduit 226 through cover 310, conduit 226 may include one or more locating, or stop, features disposed on an outer surface thereof. For example, FIG. 8 illustrates an embodiment of conduit 226 shown as conduit 226′. Conduit 226′ includes one or more protrusions 227′ located between one or more of the conduit sections 226a′, 226b′, and 226c′. Protrusions 227′ are structured to engage with a portion of cover 310 to act as a guide for assembling conduit 226′ to cover 310 and to maintain a position (e.g., a length) of the conduit sections relative to cover 310 during operation of device 200. Protrusions 227′ may be structured as, for example, a sleeve or band (e.g., silicone band) disposed around a portion of conduit 226′.

In addition to providing a pathway for routing conduit 226, first opening 310a and/or second opening 310a of cover 310 further provide a fluid path to allow gas from within interior 202a (generated by reaction of first reactant 215 with second reactant 217) to apply a pressure on a surface of piston 322. When the gas pressure reaches a threshold value, the pressure causes piston 322 to move axially within piston chamber 304c to advance needle 324 into the GI lumen wall or surrounding tissue. For example, referring to FIGS. 6-7, device 200 is shown in the second state when the gas within interior 202a (from reaction of first reactant 215 and second reactant 217) has reached a threshold pressure, which may be associated with a fully inflated state of balloon 202 such that elongated section 210 is substantially aligned with the GI lumen wall. The gas that pressurizes balloon 202 (as indicated by unidirectional arrows 330 in FIG. 6) is applied against a surface of piston 322 through first opening 310a and/or second opening 310b to cause protrusion 326d to overcome the interference condition with release mechanism 306. As a result, piston 322 is moved axially along longitudinal axis 302ab within piston chamber 304c toward first housing section 302a such that needle 324 is advanced through seal 308 into the GI lumen wall.

Still referring to FIGS. 6-7, the gas pressure (as indicated by unidirectional arrows 330 in FIG. 6) within interior 202a is also applied against an outer surface of container 220. However, first leg 310c remains biased against conduit 226 (as indicated by dashed line 310c′ in FIG. 7) by shaft 326 during a portion of the axial travel of needle 324, to thereby substantially impede a flow of fluid preparation 221 through conduit 226 until needle 324 has traveled a sufficient distance. For example, the portion of shaft 326 that extends through first opening 310a to bias first leg 310c and second leg 310d (as indicated by dashed line 310d″) may have a length that corresponds to a desired amount of axial travel of needle 324 (e.g., a desired penetration depth.) When needle 324 has traveled a sufficient distance, as shown in FIG. 6, shaft 326 is moved axially away from first leg 310c to unbias first leg 310c from conduit 226 and thereby allow a flow of fluid preparation 221 from container 220 through conduit 226 to needle 324. In this manner, device 200 can allow for sufficient penetration of needle 324 into the GI lumen wall or surrounding tissue thereof before delivery of fluid preparation 221, which may help to ensure delivery of substantially all of fluid preparation 221 at a desired location (e.g., depth) in a subject. In other words, device 200 can allow for sequential timing between penetrating the GI lumen wall and discharging fluid preparation 221 to substantially avoid discharging fluid preparation 221 into the lumen environment.

As shown in FIGS. 6-7, upon release of shaft 326 from first leg 310c to unbias first foot 310c′ away from conduit 226, the gas pressure (as indicated by unidirectional arrows 330) within interior 202a that is applied against container 220 can expel fluid preparation 221 through conduit 226, shaft channel 326c, needle channel 324a, and needle opening 324b into the GI lumen wall or surrounding tissue thereof. In this manner, device 200 can allow for delivery of one or more therapeutic agents contained in fluid preparation 221 into the subject's blood stream for systemic delivery.

Upon completion of delivery of fluid preparation 221, needle 324 may subsequently degrade within the GI lumen wall, or other area within the GI tract, along with one or more additional components of device 200 (e.g., housing 302, shaft 326, piston 322, cover 310). In embodiments where needle 324 is substantially non-degradable, device 200 includes a return spring or other biasing feature for retracting needle 324 within housing 302 after completing delivery of fluid preparation 221. In any of these embodiments, deflation valve 212 can release a substantial amount of the gas contained within interior 202a to allow for substantial deflation of balloon 202 and subsequent traversal of device 200 through the remainder of the GI tract to exit the anus of the subject.

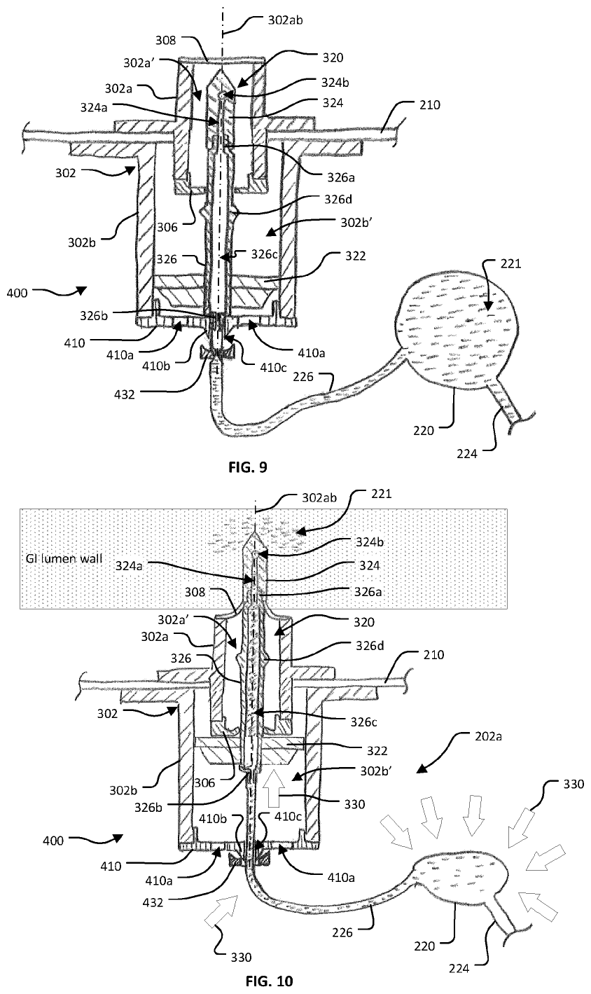

Referring to FIGS. 9-10, a delivery assembly 400 (an embodiment of delivery assembly 118) is illustrated according to another embodiment. For the sake of efficiency, the same reference numerals refer to the same components between embodiments. In this embodiment, delivery assembly 400 includes a cover 410 including a release feature 410c for interfacing with a separate valve member in the form of a clip 432 (an embodiment of valve member 116) releasably coupled to conduit 226. Release feature 410c and clip 432 may cooperatively define the valve member of this embodiment. For example, as shown in FIG. 9, cover 410 is coupled to, or integrally formed with, lower housing section 302b. Cover 410 defines one or more openings 410a to provide a fluid path between interior 202a and piston 322. Release feature 410c has a tapered (e.g., frusto-conical) structure that extends outwardly from an outer surface of cover 310. Release feature 410c further defines a conduit opening 410c′ through cover 310 for conduit 226 and/or a portion of shaft 326 to extend through. Thus, release feature 410c substantially surrounds a portion of conduit 226.

Clip 432 is releasably coupled to conduit 226 adjacent to release feature 410c. Clip 432 is coupled to conduit 226 such that conduit 226 is locally constricted by clip 432 to substantially impede a flow of fluid preparation 221 through conduit 226. Clip 432 may be structured as a C-clip, a spring clip, a claw clip, a clamp, or other structure that can be releasably coupled to conduit 226 in a similar manner. As discussed below, release feature 410c is structured to engage with clip 432 in response to sufficient axial movement of piston-needle assembly 320 so as to release clip 432 from conduit 226 and allow fluid preparation 221 to flow to needle 324.

Clip 432 may be located a sufficient distance from release feature 410c on conduit 226 to allow for sufficient penetration of needle 324 into the lumen wall before release of clip 432 by release feature 410c. For example, the distance between release feature 410c and clip 432 in the first state shown in FIG. 9 may correspond to a desired penetration depth of needle 324. In this way, delivery assembly 400 can allow for penetration of needle 324 to a desired location (e.g., depth) of the GI lumen wall or surrounding tissue thereof before delivery of fluid preparation 221.

Referring to FIG. 10, delivery assembly 400 is shown in a second state when the gas within interior 202a has reached a threshold pressure, which may be associated with a fully inflated state of balloon 202 such that elongated section 210 is substantially aligned with the GI lumen wall. The gas that pressurizes balloon 202 (as indicated by unidirectional arrows 330) is applied against a surface of piston 322 through opening(s) 410a of cover 410 to cause protrusion 326d to overcome the interference condition with release mechanism 306. As a result, piston 322 is moved axially along longitudinal axis 302ab within piston chamber 304c toward first housing section 302a such that needle 324 is advanced through seal 308 into the GI lumen wall or surrounding tissue.

The gas pressure (as indicated by unidirectional arrows 330) within interior 202a is also applied against an outer surface of container 220. However, conduit 226 remains constricted by clip 432 during a portion of the axial travel of piston-needle assembly 320 to substantially impede a flow of fluid preparation 221 through conduit 226 until needle 324 has traveled a sufficient distance. For example, as discussed above, the distance between release feature 410c and clip 432 in the first state shown in FIG. 9 may correspond to a desired amount of axial travel of needle 324 (e.g., a desired penetration depth.) When needle 324 has traveled a sufficient distance, as shown in FIG. 10, conduit 226 and clip 432 are moved axially toward release feature 410b such that release feature 410b engages with, and releases (e.g., by spreading clip 432 outwardly away from conduit 226) clip 432 from, conduit 226 to thereby allow a flow of fluid preparation 221 from container 220 through conduit 226 to needle 324.

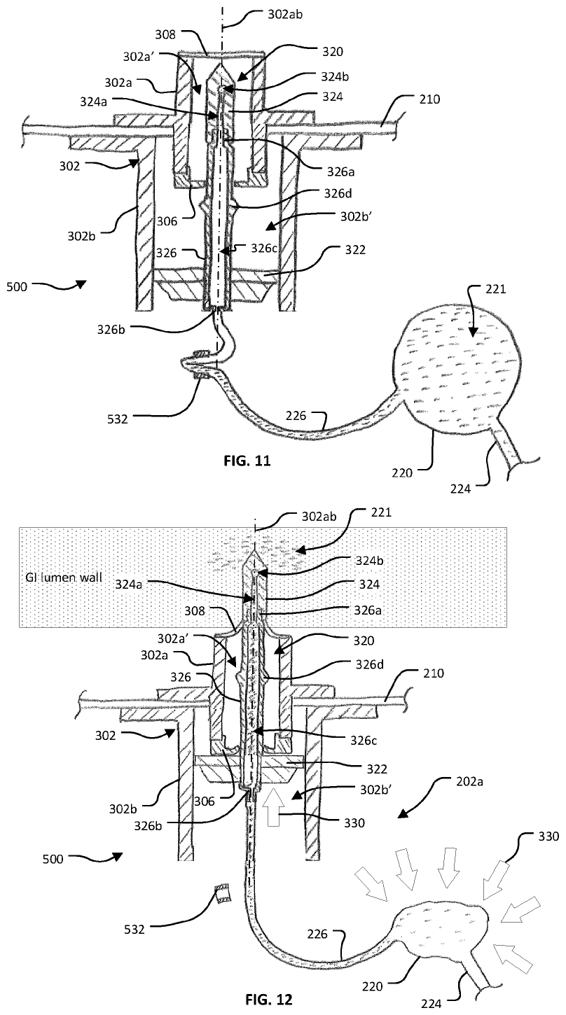

Referring to FIGS. 11-12, a delivery assembly 500 (an embodiment of delivery assembly 118) is illustrated according to another embodiment. For the sake of efficiency, the same reference numerals refer to the same components between embodiments. In this embodiment, delivery assembly 500 includes a valve member in the form of a sleeve 532 (an embodiment of valve member 116) releasably coupled to conduit 226. Sleeve 532 is coupled to conduit 226 such that a portion of conduit 226 is held in a kinked state to temporarily prevent a flow of fluid preparation 221 therethrough. In this embodiment, delivery assembly 500 is shown without a cover, however, it should be appreciated that delivery assembly 500 may include a cover according to other embodiments.

Sleeve 532 is releasably coupled around a portion of conduit 226 to hold conduit 226 in a kinked state such that conduit 226 is locally constricted to substantially impede a flow of fluid preparation 221 through conduit 226. Sleeve 532 may be structured as a flexible tube, a band, a clip, or any other structure that can be releasably coupled to conduit 226 in a similar manner. As discussed below, conduit 226 has a length such that tensioning of conduit 226 in response to sufficient axial movement of piston-needle assembly 320 causes sleeve 532 to release from conduit 226 and unkink conduit 226, to thereby allow fluid preparation 221 to flow through conduit 226 to needle 324.

Conduit 226 may have a length to allow for sufficient penetration of needle 324 into the lumen wall or surrounding tissue before release of sleeve 532. For example, the length of conduit 226 may be such that sufficient tensioning of conduit 226 to release sleeve 532 does not occur until needle 324 has traveled a sufficient axial distance, which may correspond to a desired penetration depth of needle 324. In this way, delivery assembly 400 can allow for penetration of needle 324 to a desired location (e.g., depth) of the GI lumen wall or surrounding tissue thereof before delivery of fluid preparation 221.

Referring to FIG. 12, delivery assembly 500 is shown in a second state when the gas within interior 202a has reached a threshold pressure, which may be associated with a fully inflated state of balloon 202 such that elongated section 210 is substantially aligned with the GI lumen wall. The gas that pressurizes balloon 202 (as indicated by unidirectional arrow 330) is applied against a surface of piston 322 through lower housing section 302b to cause protrusion 326d to overcome the interference condition with release mechanism 306. As a result, piston 322 is moved axially along longitudinal axis 302ab within piston chamber 304c toward first housing section 302a such that needle 324 is advanced through seal 308 into the GI lumen wall.

The gas pressure (as indicated by unidirectional arrows 330) within interior 202a is also applied against an outer surface of container 220. However, conduit 226 remains constricted by sleeve 532 during a portion of the axial travel of piston-needle assembly 320 so as to substantially impede a flow of fluid preparation 221 through conduit 226 until needle 324 has traveled a sufficient distance. For example, as discussed above, the length of conduit 226 is such that sufficient tensioning of conduit 226 to release sleeve 532 does not occur until needle 324 has traveled a sufficient axial distance (e.g., a desired penetration depth.) When needle 324 has traveled a sufficient distance, as shown in FIG. 12, conduit 226 is sufficiently tensioned to cause sleeve 532 to release from conduit 226 such that conduit 226 can unkink to allow a flow of fluid preparation 221 from container 220 through conduit 226 to needle 324.

Additionally or alternatively, conduit 226 may be formed from, or otherwise include, at least a portion made from a thermoset polymer, such as polyimide (e.g., polyimide tubing/sheet.) For example, before assembly in balloon 202, the portion of (or all of) conduit 226 including the thermoset polymer may be heated and compressed (e.g., using a mold) to create a temporary seal within conduit 226 so as to substantially impede a flow of fluid preparation 221 therethrough in a first state of the device. In this embodiment, cover 310/410, clip 432, and/or sleeve 532 may be omitted entirely and conduit 226 itself may function as the valve member (an embodiment of valve member 116.) The conduit 226 may be, but is not necessarily, held in a kinked state. In a second state, when the gas pressure applied to container 220 within interior 202a reaches a threshold value associated with delivery of fluid preparation 221 (e.g., upon needle 324 penetrating the GI lumen wall), the fluidic pressure of fluid preparation 221 in container 220 overcomes the temporary seal within conduit 226 to open conduit 226 and allow fluid preparation 221 to flow through conduit 226 and needle 324 for delivery into the GI lumen wall or surrounding tissue thereof.

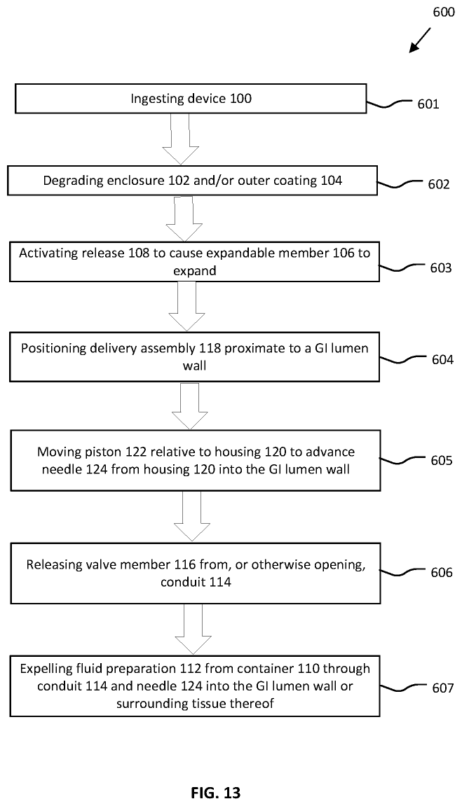

Referring now to FIG. 13, a method 600 of delivering a fluid preparation 112 into the GI tract of a subject using device 100 is illustrated. In a first step, the subject ingests device 100 (e.g., by swallowing device 100) (Step 601.) As a result of ingestion, enclosure 102 and/or outer coating 104 are at least partially (or may be fully) degraded when device 100 reaches a desired location in the GI tract for delivery of fluid preparation 112 (Step 602.) In response, release 108 is activated to cause a chemical reaction resulting in the formation of a gas within expandable member 106 to thereby cause expandable member 106 to expand within a lumen of the GI tract (Step 603.) As a result, delivery assembly 118 is positioned proximate to the GI lumen wall (Step 604.) Upon the gas pressure within expandable member 106 reaching a threshold value, piston 122 is moved relative to housing 120 to advance needle 124 into the GI lumen wall or surrounding tissue thereof (Step 605.) Upon sufficient axial movement of needle 124, valve member 116 is released from, or otherwise opens, conduit 114 (Step 606.) The gas pressure within expandable member 106 is applied against container 110 to cause container 110 to expel fluid preparation 112 through conduit 114 and needle 124 into the GI lumen wall or surrounding tissue thereof (Step 607.) In this manner, device 100 can allow for one or more therapeutic agents contained in fluid preparation 112 to be released into the subject's blood stream for systemic delivery.

The foregoing description of various embodiments has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric and neonatal applications as well as various veterinary applications. Also, those skilled in the art will recognize, or be able to ascertain using no more than routine experimentation, numerous equivalents to the specific devices and methods described herein. Such equivalents are considered to be within the scope of the present disclosure.

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure as defined by the appended claims. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

1. A delivery assembly for an ingestible device for delivering a fluid preparation into a GI lumen wall or surrounding tissue thereof of a subject, the delivery assembly comprising:
a housing defining a piston chamber;
a piston movably disposed in the piston chamber;
a needle coupled to, or integrally formed with, the piston;
a conduit structured to direct the fluid preparation from a fluid source to the needle; and
a valve member having a first state and a second state;
wherein in the first state, the valve member substantially impedes a flow of the fluid preparation from the conduit to the needle; and
wherein in the second state, the valve member is structured to allow the fluid preparation to flow through the conduit to the needle.

2. The delivery assembly of claim 1, wherein the valve member is releasably coupled to the conduit, and wherein the valve member is structured to release from the conduit in response to movement of the piston to allow the fluid preparation to flow through the conduit to the needle in the second state.

3. The delivery assembly of claim 1, wherein at least a portion of the conduit defines the valve member, and wherein the conduit is structured to open in response to a pressure of the fluid preparation to allow the fluid preparation to flow through the conduit to the needle in the second state.

4. The delivery assembly of claim 1, wherein the delivery assembly is structured to be coupled to, and disposed in, an expandable member.

5. The delivery assembly of claim 1, wherein the delivery assembly is structured to be contained in an ingestible enclosure for delivery into the GI tract of the subject.

6. The delivery assembly of claim 1, wherein the piston is structured to move in response to a gas pressure applied to an outer surface of the piston to advance the needle from the housing into the GI lumen wall while the valve member is in the first state.

7. The delivery assembly of claim 1, wherein the valve member includes a cover coupled to the housing, and wherein the cover defines an opening and a flexible leg extending into the opening.

8. The delivery assembly of claim 7, wherein the piston includes a shaft, and wherein a portion of the conduit and a portion of the shaft each extend through the opening of the cover such that the shaft biases the flexible leg against the conduit to substantially impede the flow of the fluid preparation through the conduit in the first state.

9. The delivery assembly of claim 8, wherein the piston moves relative to the housing to cause the shaft to move away from the cover to unbias the flexible leg away from the conduit to thereby allow the fluid preparation to flow to the needle in the second state.

10. The delivery assembly of claim 1, wherein the valve member includes a clip releasably coupled to the conduit, and wherein the clip substantially impedes the flow of the fluid preparation through the conduit in the first state.

11. The delivery assembly of claim 10, further comprising a cover coupled to the housing, wherein the cover defines an opening and a release feature.

12. The delivery assembly of claim 11, wherein the piston includes a shaft, wherein a portion of at least one of the conduit or the shaft extends through the opening of the cover, and wherein the clip is positioned outside of the piston chamber adjacent the release feature.

13. The delivery assembly of claim 12, wherein the piston moves relative to the housing to cause the clip to move toward the cover such that the release feature releases the clip from the conduit to thereby allow the fluid preparation to flow to the needle in the second state.

14. The delivery assembly of claim 1, wherein the valve member includes one of a sleeve, a clip, or a band releasably coupled to the conduit such that a portion of the conduit is temporarily held in a kinked state to substantially impede the flow of the fluid preparation through the conduit in the first state.

15. The delivery assembly of claim 14, wherein the piston moves relative to the housing to cause tensioning of the conduit such that the sleeve, clip, or band is released from the conduit to thereby allow the fluid preparation to flow to the needle in the second state.

16. The delivery assembly of claim 1, wherein at least a portion of the conduit defines the valve member, and wherein the at least a portion is formed from a thermoset polymer which defines a temporary seal within the conduit to substantially impede the flow of the fluid preparation through the conduit in the first state.

17. The delivery assembly of claim 16, wherein a pressure of the fluid preparation overcomes the temporary seal to thereby allow the fluid preparation to flow through the conduit to the needle in the second state.

18-43. (canceled)

44. A delivery assembly for an ingestible device to deliver a fluid preparation into a GI lumen wall or surrounding tissue thereof of a subject, the delivery assembly comprising:
a housing defining a piston chamber;
a piston movably disposed in the piston chamber;
a needle coupled to the piston; and
a valve member comprising a cover coupled to the housing;
wherein the piston is structured to be coupled to a conduit and a container including the fluid preparation;
wherein the valve member is structured to permit a flow of the fluid preparation through the conduit and piston to the needle in response to axial movement of the piston relative to the housing.

45. A delivery assembly for an ingestible device to deliver a fluid preparation into a GI lumen wall or surrounding tissue thereof of a subject, the delivery assembly comprising:
a housing defining a piston chamber;
a piston movably disposed in the piston chamber; and
a needle coupled to the piston;
wherein the piston is structured to be coupled to a conduit and a container including the fluid preparation;
wherein the needle is structured to move axially relative to the housing via the piston to penetrate the GI lumen wall independent of the fluid preparation.

46. The delivery assembly of claim 44, wherein the needle is structured to move axially relative to the housing in response to a pressure applied to an outer surface of the piston.
